(19) United States
(12) Patent Application Publication
(10) Pub. No.: US 2026/0202259 A1
(43) Pub. Date: Jul. 16, 2026

(54) INSPECTION DEVICE, INSPECTION SYSTEM, NON-TRANSITORY COMPUTER-READABLE STORAGE MEDIUM, INSPECTION METHOD, AND MANUFACTURING METHOD

(71) Applicant: NIPPON AVIONICS CO., LTD., Yokohama-shi (JP)

(72) Inventor: Ryuji SEKIMOTO, Yokohama-shi (JP)

(21) Appl. No.: 19/433,454

(22) Filed: Dec. 26, 2025

(30) Foreign Application Priority Data
Jan. 16, 2025 (JP) 2025-006077

(51) Int. Cl. G01K 7/02; G01N 21/956

(52) U.S. Cl. CPC G01K 7/02; G01N 21/956; G01N 2021/95646

(57) ABSTRACT

An inspection device is configured to inspect quality of solder joining a first component and a second component. The inspection device comprises a detection unit configured to detect, by a heat flux sensor, a heat flux corresponding to a heat flow from the first component through the solder and then through the second component as a result of heating the first component at a temperature that does not melt the solder; and an acceptability determination unit configured to determine whether the quality is acceptable or unacceptable based on the heat flux detected by the detection unit.

### TECHNICAL FIELD

The present disclosure relates to an inspection device, an inspection system, a non-transitory computer-readable storage medium, an inspection method, and a manufacturing method.

### BACKGROUND

As disclosed in Patent Document 1 (JP 2010-56188 A), soldering is known in which a first component and a second component are joined with solder.

Solder (including brazing filler material) may contain voids after soldering. High void content can cause poor joining between the first and second components (in particular, poor electrical connection). In other words, solder with a high void content is of poor quality (i.e., unacceptable). Since it is difficult to completely eliminate voids, it is preferable to inspect the quality of solder in soldered products after soldering.

### SUMMARY

An object of the present disclosure is to accurately inspect the quality of solder that joins a first component and a second component.

An inspection device according to the present disclosure is an inspection device configured to inspect quality of solder joining a first component and a second component, the inspection device comprising: a detection unit configured to detect, by a heat flux sensor, a heat flux corresponding to a heat flow from the first component through the solder and then through the second component as a result of heating the first component at a temperature that does not melt the solder; and an acceptability determination unit configured to determine whether the quality is acceptable or unacceptable based on the heat flux detected by the detection unit.

An inspection system according to the present disclosure is an inspection system comprising: the inspection device; a heater tool configured to heat the first component by generating heat while contacting the first component; and the heat flux sensor configured to contact the second component.

A non-transitory computer-readable storage medium according to the present disclosure is a non-transitoy computer-readable storage medium storing a program that, when executed by a computer for inspecting quality of solder j oining a first component and a second component, causes the computer to perform: a heating step of heating the first component at a temperature that does not melt the solder; a detection step of detecting, by a heat flux sensor, a heat flux corresponding to a heat flow from the first component through the solder and then through the second component as a result of the heating step; and a determination step of determining whether the quality is acceptable or unacceptable based on the heat flux detected by the detection step.

An inspection method according to the present disclosure is a method for inspecting quality of solder joining a first component and a second component, the method comprising the steps of a heating step of heating the first component at a temperature that does not melt the solder; a detection step of detecting, by a heat flux sensor, a heat flux corresponding to a heat flow from the first component through the solder and then through the second component as a result of the heating step; and a determination step of determining whether the quality is acceptable or unacceptable based on the heat flux detected by the detection step.

A manufacturing method according to the present disclosure is a manufacturing method comprising the steps of: a first step of producing a soldered product including the first component, the second component, and the solder by soldering the first component and the second component together with the solder; and a second step of inspecting quality of the solder in the produced soldered product by the inspection method.

In accordance with the present disclosure, the quality of the solder joining the first component and the second component can be inspected accurately.

### BRIEF DESCRIPTION OF THE DRAWINGS

FIG. 1 is a schematic diagram illustrating a configuration of a joint inspection system according to an embodiment of the present disclosure, and illustrates a state before soldering.

FIG. 2 is a schematic diagram illustrating the configuration of the joint inspection system according to the embodiment of the present disclosure, and illustrates how a product is transported after soldering.

FIG. 3 is a schematic diagram illustrating the configuration of the joint inspection system according to the embodiment of the present disclosure, and illustrates how solder quality is inspected.

FIG. 4 is a block diagram illustrating a main configuration of a controller.

FIG. 5 is a flowchart of a soldering and quality inspection process.

FIG. 6 is a flowchart of a soldering process.

FIG. 7 is a flowchart of a quality inspection process.

FIG. 8A is a graph illustrating a temperature profile representing the change over time in the temperature of a heater tool during heating for inspecting solder quality.

FIG. 8B is a graph illustrating the change over time in the current that causes the heater tool to generate heat so as to achieve the temperature change shown in FIG. 8A.

FIG. 9 is a graph illustrating the change over time in heat flux detected by a heat flux sensor during heating for solder inspection.

### DETAILED DESCRIPTION

Hereinafter, embodiments of the present disclosure will be described with reference to the drawings. As used herein, the term "soldering" includes brazing. In other words, "solder" includes brazing filler metal/material.

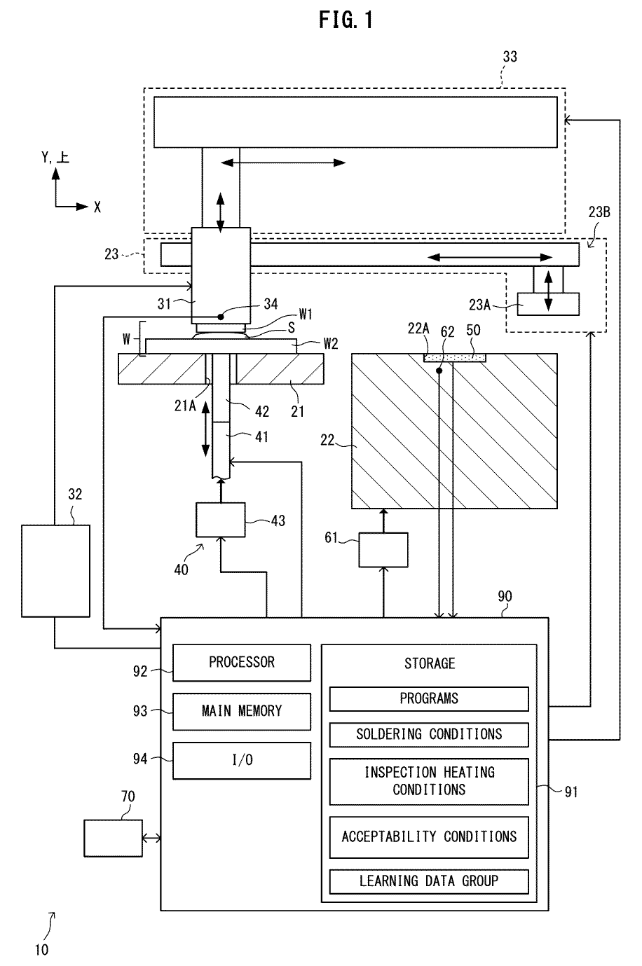

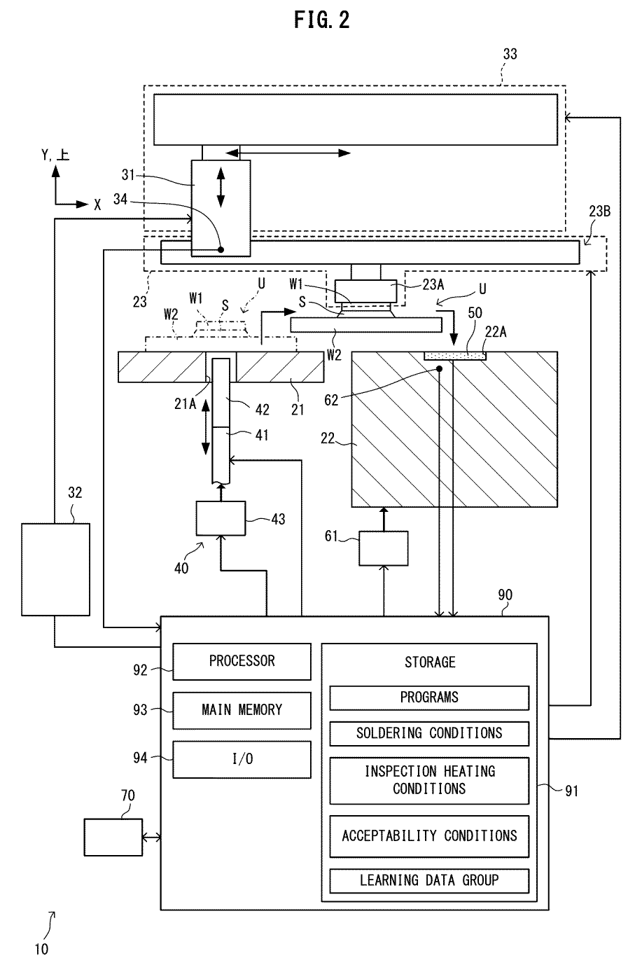

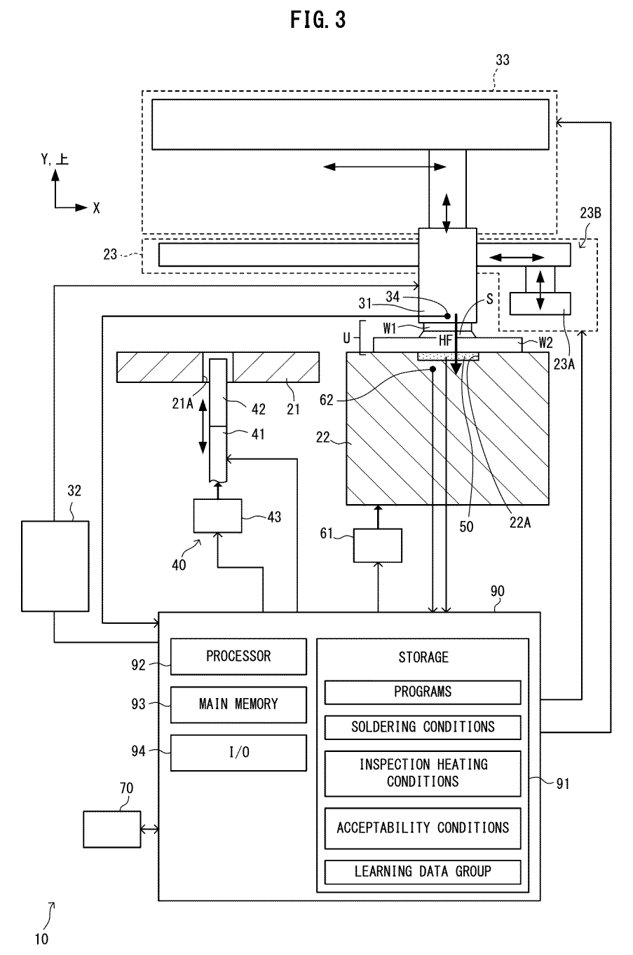

In the joint inspection system 10 according to the embodiment shown in FIGS. 1-3, a component W1 and a component W2 are soldered together with solder S. By such soldering, the solder S melts once and then solidifies, thereby joining the component W1 and the component W2. The component W1 is, for example, a power semiconductor device. The component W2 is, for example, a lead frame made of copper. The solder S may be configured as a plurality of bumps which connect a plurality of terminals of the power semiconductor device to a plurality of terminals of the lead frame, respectively. Components W1 and W2 and solder S therebetween, before being soldered, are also referred to as a "workpiece W" (see FIG. 1). A workpiece W after soldering, that is, a combination of components W1 and W2 and solder S which joins them together, is also referred to as a "product U" (see FIGS. 2 and 3). Such a product U is a product after soldering, and can therefore also be called a "soldered product". The product U may not be a final product, but may be a component used in a final product. The joint inspection system 10 inspects the quality of solder S in the product U in addition to the above-described joining (soldering).

The joint inspection system 10 includes a stage 21, a stage 22, a transport device 23, a heater tool 31, a power supply circuit 32, a driving device 33, a temperature sensor 34, an ultrasonic application device 40, a heat flux sensor 50, a cooling device 61, a temperature sensor 62, an input/output device 70, and a controller 90. In FIGS. 1 and 2, the stages 21 and 22 are shown in cross section. The X-axis and Y-axis set in FIGS. 1 and 2 extend in directions perpendicular to each other. The Y-axis direction is also called an "up-down direction". Such an up-down direction may be a direction defined for convenience, and may or may not coincide with the actual vertical direction. The stage 21 supports the workpiece W to be soldered from the side of the component W2, that is, from below (-Y-axis direction; see FIG. 1). The workpiece W is soldered on the stage 21 by the heater tool 31. The stage 21 may be provided with recesses and protrusions or a jig for positioning the workpiece W (in particular, the second component W2). The product U, which is the workpiece W that has finished the soldering process, is transported from the stage 21 to the stage 22 arranged alongside the stage 21 in the X-axis direction. This transport is performed by a transport device 23 which operates under the control of the controller 90.

The transport device 23 includes a holding member 23A for holding the product U and a driving device 23B that is constituted by a robotic device including a linear motor or a ball screw mechanism, etc., and moves the holding member 23A in at least the X-axis and Y-axis directions. The holding member 23A holding the product U is moved by the driving device 23B (see FIG. 2), thereby transporting the product U. In order to prevent interference with the heater tool 31, the driving device 23B is positioned further back than the heater tool 31 with respect to the plane of the paper in FIGS. 1- 3, and the holding member 23A extends from the driving device 23B to the front side with respect to the plane of the paper in FIGS. 1-3. The product U may be transported to the stage 22 by the user instead of by the transport device 23.

The stage 22 to which the product U is transported supports the product U from the side of the component W2, that is, from below, during inspection of the quality of the solder S in the product U (see FIG. 3). The quality of the solder S in the product U is inspected on the stage 22. During this quality inspection, the product U is heated by the heater tool 31. The stage 22 may be provided with recesses and protrusions or a jig for positioning the product U (in particular, the second component W2).

The heated tool 31 heat the workpiece W (FIG. 1) on the stage 21 for soldering, and also heats the product U (FIG. 3) on the stage 22 for inspection of the quality of the solder S. The heated tool 31 heats the component W1 from above (+Y-axis direction), thereby heating the work piece W or product U from the side of the component W1, ie., from above. The heater tool generates heat by means of a current from the power supply circuit 32 to perform the heating. The heater tool 31 can be a typical heater tool used for soldering. The heater tool 31 can include a heater tip that locally generates heat at a portion that comes into contact with workpiece W or the product U when the above-described current flows through it. The heater tool 31 can be moved by the driving device 33.

The power supply circuit 32 includes an inverter circuit, etc. Under the control of controller 90, the power supply circuit 32 supplies a current to the heater tool 31 to cause the heater tool 31 to generate heat, and controls the amount of the current.

The driving device 33 is constituted by, for example, a robotic device including a linear motor, a ball screw mechanism, etc., and moves the heater tool 31 at least in the X-axis direction and the Y-axis direction under the control of controller 90. The driving device 33 moves the heater tool 31 in the X-axis direction to move it between a position above the stage 21 and a position above the stage 22. During soldering, the driving device 33 moves the heater tool 31, which is located above the stage 21, downward to bring it into contact with component W1 of the workpiece W on the stage 21 from above (FIG. 1). During quality inspection of the solder S in the product U, the driving device 33 moves the heater tool 31 located above the stage 22 downward to bring it into contact with the component W1 of the product U on the stage 22 from above (FIG. 3). When the heater tool 31 is in contact as described above in both cases, the driving device 33 preferably presses the heater tool 31 against the component W1. Such pressing applies pressure to the workpiece W and the product U from above and below (Y-axis direction, or thickness direction).

The heater tool 31 is equipped with a temperature sensor 34. The temperature sensor 34 is made of a thermocouple and converts the temperature of the heater tool 31 into electric signals. The converted electrical signals are supplied to the controller 90. The controller 90 performs predetermined processing on the electrical signals to derive the temperature of the heater tool 31, thereby detecting the temperature of the heater tool 31. The detected temperature is used in the controller 90 to control the heat generation temperature of the heater tool 31 (control the current to the heater tool 31).

The ultrasonic application device 40 applies ultrasonic waves to the workpiece W during soldering. The ultrasonic application device 40 includes an ultrasonic oscillator (also simply referred to as an "oscillator") 41, an ultrasonic hom (also simply referred to as a "hom") 42, and a driving device 43. The oscillator 41 is, for example, a Langevin type oscillator, and emits ultrasonic wave. The hom 42 is fixed to the oscillator 41 and inserted into a through-hole 21A formed of the stage 21. The hom 42 amplifies ultrasonic wave generated by the oscillator 41. The driving device 43 is constituted by a linear motor, a ball screw mechanism, etc. The driving device 43 moves the oscillator 41 and the hom 42 upward, and presses the tip of the hom 42 against the component W2 of the workpiece W on the stage 21. During the pressing, the oscillator 41 generates ultrasonic wave. The generated ultrasonic wave is amplified by the hom 42 and applied to the workpiece W from the side of component W2. The operation of the ultrasonic application device 40, specifically, the operation of the driving device 43 and the vibration of the oscillator 41, are controlled by the controller 90.

The heat flux sensor 50 is positioned and housed in a recess 22A which open to the upper surface of the stage 22. In this way, the stage 22 positions and support the heat flux sensor 50. for the purpose of ensuring visibility, schematic diagram of the heat flux sensor 50 are shown with dot patters in FIGS. 1-3. In order to come in contact with the component W2 of the product U, the upper surface of the heat flux sensor 50 is flush with the upper surface of the stage 22 or is positioned higher than the upper surface of the stage 22. In the latter case, the stage 22 supports hte product U via the heat flux sensor 50.

When inspecting the quality of the solder S in the product U, the heatertool 31 applies pressure and heat to the product U on the stage 22. By applying pressure with the heater tool 31, the product U is sandwiched between at least the heat flux sensor 50 and the heater tool 31. As a result, at least the heat flux sensor 50 is pressed against the component W2 of the product U. The product U is heated by the heater tool 31 from the side of the component W1. The heat generation temperature of the heater tool 31, that is, the heating temperature at which the product U (more specifically, the component W1) is heated, is set to a temperature at which the solder S does not melt, since this is for quality inspection of the solder S.

Heating by the heater tool 31 generates a heat flow HF (FIG. 3) in the product U, which flows from the component W1 through the solder S to the component W2. The heat flow HF flows from the component W2 through the heat flux sensor 50 to the stage 22. The heat flux sensor 50 converts the heat flux q [W/m²] corresponding to the heat flow HF into an electrical signal (here, a voltage signal). The heat flux sensor 50 provides the converted electrical signal indicative of the heat flux q to the controller 90. The controller 90 performs predetermined processing on the electrical signal to derive the heat flux q, thereby detecting the heat flux q. In this way, the controller 90 detects, by the heat flux sensor 50, the heat flux q corresponding to the heat flow HF that flows from the component W1 to the solder S and then flows to the component W2. The controller 90 inspects the quality of the solder S based on the hear flux q. The heat flux sensor 50 is, for example, a plate-shaped sensor, specifically Toyota Motor Corporation's "Energy Flow" (registered trademark).

The stage 22 is heated by heating the product U with the heater tool 31. Even if the stage 22 is heated as described above, it is cooled by the cooling device 61 and kept at a constant temperature. The cooling device 61 may be either an air-cooling type or a water-cooling type. The air-cooling type cooling device 61 may be a blower such as a fan. The cooling device 61 may be a cooling device using a Peltier element. The cooling device 61 is controlled by a controller 90.

The stage 22 is provided with a temperature sensor 62 for converting the temperature at a portion of the stage 22 in the vicinity of the heat flux sensor 50 into an electrical signal. The temperature sensor 62 is constituted by, for example, a thermocouple. The electrical signal converted by the temperature sensor 62 is supplied to the controller 90. The controller 90 performs predetermined processing on the electrical signal to derive the temperature of the stage 22, thereby detecting the temperature of stage 22. The detected temperature is used in the controller 90 to control the temperature of the stage 22, that is, to control the cooling device 61.

The input/output device 70 includes a display device, an input device, etc., and displays various images and accepts various operations from the user.

The controller 90 is constituted by various types of computers and includes a non-volatile storage 91 for storing programs, etc., and a processor92 for executing the programs stored in the storage 91. The storage 91 also stores data used in the processes described later, such as soldering condition, inspection heating condition, acceptability condition, and learning data group. The processor92 includes one or more central processing units (CPUs). The controller 90 further includes a main memory 93 for providing a working area for the processor 92; and an input/output (I/O) 94 for relaying electrical signals sent and received between the processor 92 and the outside of the controller 90. The I/O 94 may include a circuit for performing amplification and analog-to-digital conversion of electrical signals from the temperature sensor 34, electrical signals from the heat flux sensor 50, electrical signals from the temperature sensor 62, and manipulation signals from the input/output device 70. The I/O 94 may also include a circuit for performing digital-to-analog conversion of the control signals from the processor 92 and supplying the converted signal to devices (e.g., the transport device 23, the power supply circuit 32, the driving device 33, the ultrasonic application device 40, the cooling device 61, and the input/output device 70) to be controlled.

The soldering condition specifies the control mode of the power supply circuit 32 during soldering. The inspection heating condition specifies the control mode of the power supply circuit 32 during inspection of the quality of the solder S in the product U. The specific details of both conditions are arbitrary, but each condition here includes a temperature profile which indicates the change over time in the heat generation temperature of the heater tool 31, i.e., the heating temperature of the workpiece W or product U (more specifically, component W1). The acceptability condition includes one or more thresholds used during inspection of the quality of the solder S. The respective condition can be set or changed by the user operating the input/output device 70. The learning data group includes a plurality of items of learning data. Each item of learning data includes a quality inspection result and a soldering condition corresponding to the quality, which are associated with each other.

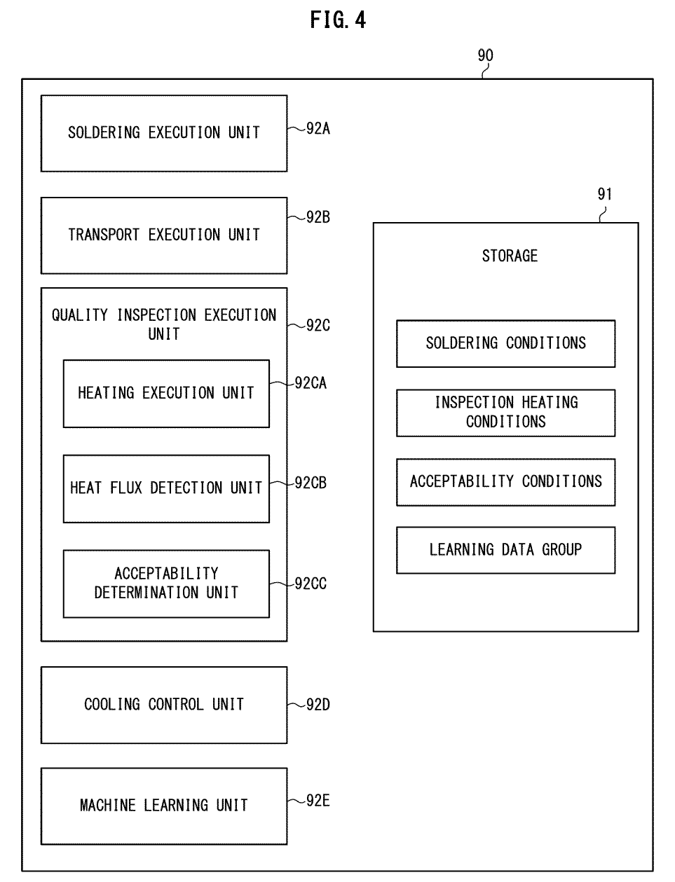

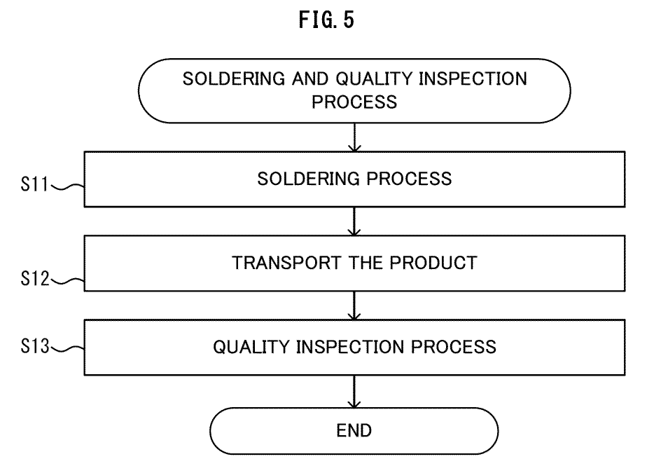

The processor 92 executes programs stored in the storage 91 to operate as a soldering execution unit 92A, a transport execution unit 92B, a quality inspection execution unit 92C, a cooling control unit 92D, and a machine learning unit 92E shown in FIG. 4. The quality inspection execution unit 92C includes a heating execution unit 92CA, a heat flux detection unit 92CB, and an acceptability determination unit 92CC. The units 92A to 92C cooperate to carry out the soldering and quality inspection process shown in FIG. 5. The soldering and quality inspection process is performed for each of a plurality of workpieces W in sequence. In this way, a plurality of products U are produced.

In the soldering and quality inspection process, first, the soldering execution unit 92A performs a soldering process for soldering the workpiece W places on the stage 21 (step S11). The workpiece W is placed on the stage 21 by a transport mechanism (not illustrated) or by a user.

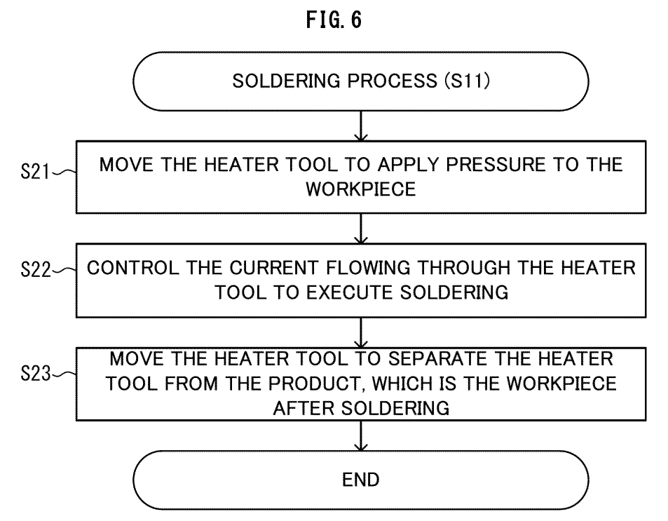

A detailed example of the soldering process will be described with reference to FIG. 6. First, the soldering execution unit 92A controls the driving device 33 to move the heater tool 31 above the stage 21, and then moves it to downward to apply pressure to the workpiece W (step S21; see the state in FIG. 1). The workpiece W is sandwiched between the stage 21 and the heater tool 31 thus moved and is pressed in the thickness direction.

After step S21, the soldering execution unit 92A controls the power supply circuit 31 in accordance with the soldering condition stored in the storage 91, thereby controlling the current flowing through the heater tool 31 and executing soldering (step S22). The soldering execution unit 92A may start step S22 when it is detected that the pressure, which is detected using a pressure sensor (not illustrated) installed on a suitable position, applied to the workpiece W exceeds a predetermined pressure. in step S22, first, the heater tool 31 generates heat, and the component W1 in contact with the heater tool 31 is heated. The entire workpiece W is heated by heat conduction caused by this heating. As a result of the workpiece W being heated, the solder S melts. After a certain period of time has elapsed, the current is controlled so as to terminate the heating, and after the heating is terminated, the solder S hardens. By such series of operations in step S22, the soldering of the components W1 and W2 is executed, joining the components W1 and W2 together with the solder S. Such joining causes a product U, which is the workpiece W after soldering, to be produced.

In step S22, the soldering execution unit 92A periodically detects the temperature of the heater tool 31 (more specifically, the heat generation temperature) using the temperature sensor 34. Based on the detected temperature, the soldering execution unit 92A feedback controls the power supply circuit 32 (e.g., the switching frequency of the switching element of the inverter circuit) such that the change over time in the heat generation temperature of the heater tool 31 matches the temperature profile included in the soldering condition. Here, the temperature profile uses a pulse heat profile that increases the temperature of the heater tool 31 in a pulsed (i.e., instantaneous) manner. This allows heating or soldering to be performed using a pulse heat method, and the heating time for the workpiece W can be shortened.

During soldering in step S22, the soldering execution unit 92A controls the ultrasonic application device 40 to apply ultrasonic wave to the workpiece W. In particular, the soldering execution unit 92A ultrasonically vibrates the oscillator 41 and controls the driving device 43 to move the oscillator 41 and the hom 42 upward, and presses an end of the horn 42 against the workpiece W from below. As a result, ultrasonic wave is applied to the workpiece W. The application of the ultrasonic wave reduces the volume of voids that are formed in the solder S during soldering.

After step 22, the soldering execution unit 92A controls the driving device 33 to move the heater tool 31 upward and separate the heater tool 31 from the product U, which is the workpiece W after soldering (step S23; see the state in FIG. 2). At this time, the soldering execution unit 92A controls the ultrasonic application device 40 also to move the hom 42 away from the workpiece W and stop the ultrasonic wave oscillation.

Referring again to FIG. 5, after step S11, the transport execution unit 92B controls the transport device 23 to transfer the product U on the stage 21 to the stage 22 (step S12). Thereafter, the quality inspection execution unit 92C executes the quality inspection process (step S13).

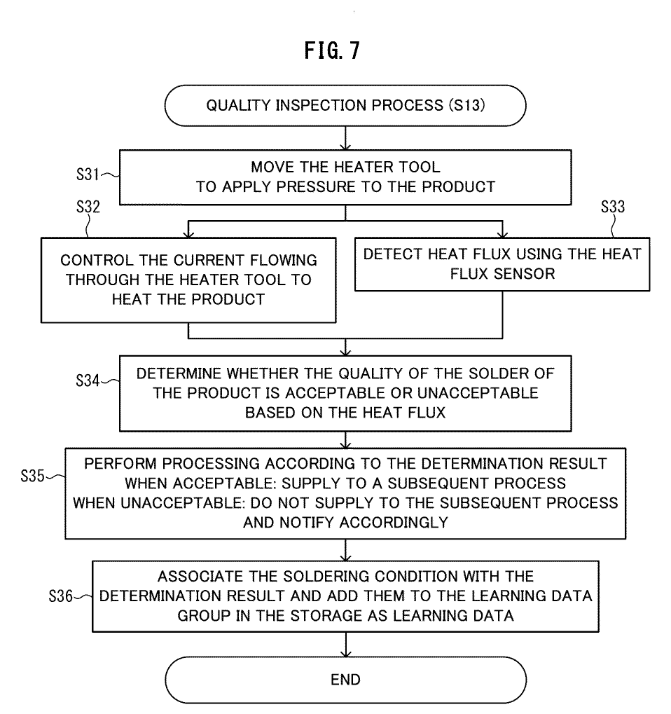

The quality inspection execution unit 92C executes, for example, the quality inspection process shown in FIG. 7. In this process, first, the heating execution unit 92CA of the quality inspection execution unit 92C controls the driving device 33 to move the heater tool 31 above the stage 22, and then move it downward, thereby applying pressure to the product U on the stage 22 (step S31; see the state in FIG. 3). The product U is sandwiched between at least the heat influx sensor 50 (of the stage 22 and the heat flux sensor 50), and the heater tool 31, and is pressed in the thickness direction. This pressure causes the heat flux sensor 50 in contact with the component W2 of the product U to be pressed against the component W2 and come into close contact with it.

After step S31, the heating execution unit 92CA controls the power supply circuit 32 according to the inspection heating condition stored in the storage 91, thereby controlling the current flowing through the heater tool 31 and heating the product U (step S32). The heating execution unit 92CA may start step S32 when it is detected that the pressure applied to the product U exceeds a predetermined pressure, as detected by a pressure sensor (not illustrated) provided at a predetermined position.

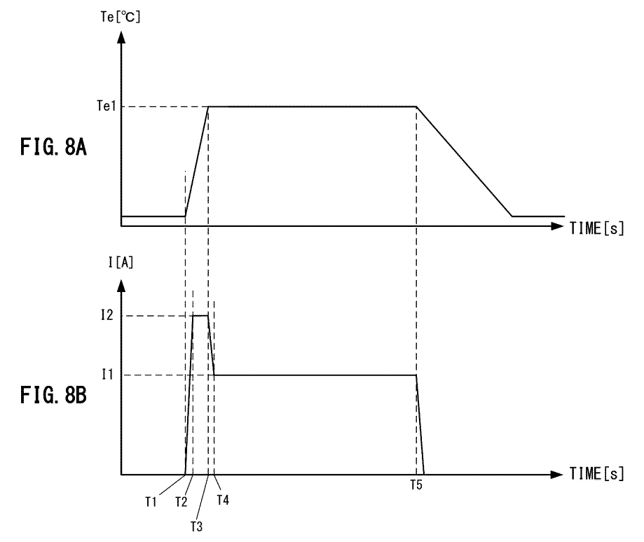

In step S32, the heating execution unit 92CA feedback controls the power supply circuit 32 based on the temperature (heat generation temperature) of the heater tool 31 detected by the temperature sensor 34 such that the change over time in the heat generation temperature of the heater tool 31 matches the temperature profile included in the inspection heating condition. The temperature profile here is a pulse heat profile that increases the temperature of the heater tool 31 in a pulsed manner. As shown in FIG. 8A, for example, this temperature profile raises the temperature Te of the heater tool 31 to a predetermined temperature Te1 (a temperature lower than the melting temperature of the solder S) in a short time (e.g., a few seconds) (T1 to T4), then holds the predetermined temperature Te1 for a predetermined period (e.g., a few seconds to several tens of seconds) (T4 to T5), and then lowers it. As shown in FIG. 8B, the heating execution unit 92CA suddenly increases the current that causes the heater tool 31 to generate heat to a current I2 that is larger than the current I1 that causes the heater tool 31 to heat a predetermined temperature Te1, and maintains this current (T1 to T3). When the temperature Te detected by the temperature sensor 34 reaches the temperature Te1, the heating execution unit 92CA reduces the current supplied to the heater tool 31 to the above-described current I1 (T3 to T4), maintains the current I1 for a predetermined period (T4 to T5), and then terminates the supply of the current to the heater tool 31.

Heating of the product U by the heater tool 31 in step S32 generates the heat flow HF (FIG. 3) that flows from the heater tool 31 to the stage 22 via the product U and the heat flux sensor 50. The heat flux detection unit 92CB uses the heat flux sensor 50 to detect the heat flux q corresponding to the heat flow HF passing through the heat flux sensor 50, i.e., the heat flux q corresponding to the heat flow HF that flows from component Wi of the product U through the solder S and then through component W2 (step S33). The above-described detection is performed periodically. The periodically detected values of heat flux q are stored in the main memory 93 in chronological order as time-series data. Step S33 is performed in parallel with step S32. The time-series data is data on the change over time in the heat flux q.

When step S32 ends, step S33 also ends. After steps S32 and S33 are ended, the acceptability determination unit 92CC determines the acceptability of the solder S in the product U based on the time- series data of the heat flux q detected by the heat flux detection unit 92CB and stored in the main memory 93 (step S34).

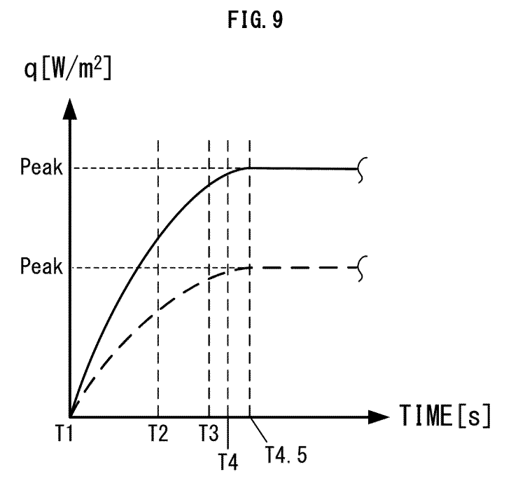

As shown in the graph in FIG. 9 (solid and dashed lines), during the heating, the heat flux q rises rapidly for a predetermined period immediately after the heater tool 31 starts heating, for example, a period from timing T1 when the heating starts until timing T4 when the temperature of the heater tool 31 reaches a predetermined temperature Tel, which heats the product U. Thereafter, the heat flux q reaches the peak value (from timing T4.5 onward). Since the stage 22 is kept at a constant temperature by the cooling device 61, the peak value continues until the heating of the heater tool 31 ends. If no cooling is performed by the cooling device 61, the heat flux q gradually decreases after reaching the peak due to heat conduction.

Of the two curves in FIG. 9, the solid line indicates a case where the volume of voids in the solder S is small and the quality of the solder S is acceptable, whereas the dashed line indicates a case where the volume of voids is large and the quality of the solder S is unacceptable. As is clear from both curves in FIG. 9, the increase in heat flux q becomes smaller when the volume of voids is large. This is because the voids have a heat insulating effect. The solid line curve and the dashed line curve differ in the rate of increase of the heat flux q immediately after the start of heating, more specifically, during a period from the start of heating until the temperature Te of the heater tool 31 reaches the temperature Tel. Such a rate of increase can be expressed by the gradient, etc., of the change over time in the heat flux q. Furthermore, the solid line curve and the dashed line curve also differ in the value of the heat flux q at a predetemined timing, particularly when the heat flux q reaches its peak.

In step S34, the acceptability determination unit 92CC determines that the quality of the solder S is acceptable when the rate of increase of the heat flux q immediately after the start of heating is greater than or equal to a threshold Th1, and determines that the quality of the solder S is unacceptable when the rate of increase is less than the threshold Th1. Alternatively, or additionally, in step S34, the acceptability determination unit 92CC determines that the quality of the solder S is acceptable when the value of the heat flux q at the above-described predetermined timing is greater than or equal to the threshold Th2, and determines the quality of the solder S is unacceptable when the value is less than the threshold Th2. In step S34, the acceptability determination unit 92CC may determine the quality of the solder S is acceptable when the rate of increase of the heat flux q immediately after the start of heating is greater than or equal to a threshold Th1 and the value of the heat flux q at the predetermined timing is greater than or equal to a threshold Th2, and determine that the quality of the solder S is unacceptable if this is not the case.

In addition, the acceptability determination unit 92CC may compare the pattern of change over time in the heat flux q indicated by the time-series data of the heat flux q (for example, a graph shown in FIG. 9 based on the time-series data) with a reference pattern (for example, a reference graph) which is the pattern of change over time in the heat flux when the quality of the solder S is acceptable, prepared in advance through experiments, etc. The acceptability determination unit 92CC determines that the quality of the solder S is unacceptable when the magnitude of the deviation between the two patterns (more specifically, the deviation in the direction of smaller heat flux q) is equal to or greater than a predetermined criterion. An example of the case where the magnitude of the deviation is equal to or greater than the predetermined criterion is when the similarity between both patterns (for example, images of graphs) is less than a specific criterion. In addition, it may be possible to adopt a case where the above-described reference pattern is set to a pattern having a width set as a range of acceptable values of heat flux (for example, a graph in a form of band curve), and the pattern of change over time in the heat flux indicated by the time-series data of heat flux q deviates by a predetermined amount or more from the reference pattern having the above-described width. If the magnitude of the deviation between the two patterns is less than a predetermined criterion, the acceptability determination unit 92CC determines that the quality of the solder S is acceptable.

The above threshold Th1, threshold Th2, reference patter and the respective reference amounts are included in the acceptability condition stored in the storage 21 and are referred to by the acceptability determination unit 92CC.

After step S34, the acceptability determination unit 92CC performs processing according to the determination result (step S35). For example, when the acceptability determination unit 92CC determines that the product U is acceptable, it performs a process to supply the product U to a subsequent process (more specifically, to a device that performs subsequent processing). For example, the acceptability determination unit 92CC controls a transport device (not illustrated) to transport the product U on the stage 22 to a subsequent process. The subsequent process includes a packaging process (e.g., a packaging device) when the product U is a final product. If the product U is not a final product, the subsequent process includes the next processing device or assembly device. The subsequent process may be done manually. In this case, the acceptability determination unit 92CC displays on the input/output device 70 that the product U is an acceptable product such that it can be supplied to a subsequent process. If the acceptability determination unit 92CC determines that the product U is defective, it treats the product U as an unacceptable product and does not supply it to the subsequent process. Treating the product U as unacceptable product involves doing the soldering of the components W1 and W2 for the unacceptable product U. In this case, the acceptability determination unit 92CC may control the transport device 23 to return the product U to the stage 21 and perform the soldering using the heater tool 31 again. treating the product U as an unacceptable product involves the acceptability determination unit 92CC controlling the device (not illustrated) to transport the unacceptable product U for disposal. Treating the product U as an unacceptable product involves the acceptability determination unit 92CC informing the user that the product is defective by displaying the inspection result on the input/output device 70, for example.

The acceptability determination unit 92CC associates the soldering condition used in step S11 at this time with the determination result, i.e., the inspection result, obtained in step S34 at this time and adds them to the learning data group in the storage 91 as learning data (step S36).

After a series of process like the above, the soldering and quality inspection process ends.

Heating the solder S in the product U during quality inspection causes the stage 22 to be heated. In this embodiment, since the heat generation temperature (in other words, the target temperature) of the heater tool 31 during inspection is fixed, the magnitude of heat flux q changes depending on the temperature of stage 22. Therefore, even if pieces of solder S to be inspected have the same volume of voids, different heat fluxes q may may be obtain depending of the temperature of stage 22 at that time. Accordingly, it is preferable that the temperature of the stage 22 be kept constant. Therefore, the cooling control unit 92D in FIG. 4 maintains the temperature of the stage 22 at a desired temperature by controlling the cooling device 61 using feedback control with the temperature of the stage 22 detected by the temperature sensor 62 shown in FIGS. 1-3 as the feedback value. This also keeps the temperature of the heat flux sensor 50 at a desired temperature. In order to facilitate the generation of the heat flow HF and the above-described temperature control, the stage 22 is preferably made of a material with good thermal conductivity, such as copper.

In addition, since the temperature of the stage 22 only needs to be at the desired temperature immediately before the quality inspection, the cooling control unit 92D may operate the cooling device 61 when the temperature of the stage 22 detected by the temperature sensor 62 has not reached the desired temperature, at least before the quality inspection. The stage 22 may also be cooled naturally. In this case, the cooling device 61, the temperature sensor 62, and the cooling control unit 92D are unnecessary.

The machine learning unit 92E in FIG. 4 operates at any timing, performs machine learning based on a learning data group consisting of learning data accumulated in the storage 91, and updates the soldering condition stored in the storage 91. Machine learning with correct answers is performed, using the inspection result from the learning data as correct answer data. Whether or not to perform such machine learning is optional, but if machine learning is performed, the soldering condition used during soldering may be changed automatically or manually each time soldering is performed one or more times. This provides a large amount of training data that is useful for machine learning. Machine learning updates the soldering condition such that the inspection results are more likely to be acceptable.

During soldering, only the component W2 may be placed on the stage 21, and the heater tool 31 may hold the component W1 and move it onto the component W2. This retention is done by, for example, suction. In this case, the heater tool 31 is provide with a suction port for suctioning the component W2. The heater tool 31 also includes pump for performing suction through the suction port. This pump may be fixed directly to the heater tool body, which is the part of the heater tool 31 that has the suction port and generates heat, or it may be positioned separately from the heater tool body and connected to the heater tool body by a tube. When the heater tool 31 holds the component W1, the solder S may be placed on either the component W2 or the component W1. If the heater tool 31 has such function, the product U may be held by the heater tool 31 and transported from the stage 21 to stage 22 by moving the heater tool 31 using the driving device 33. In this case, the transport device 23 is not necessary. Even in this configuration, the stage 21 or 22 can be said to support the entire workpiece W or the entire product U from below during soldering or quality inspection.

As described above, the controller 90 operates as an inspection device for inspecting the quality of the solder S that joins the component W1 and W2. The controller 90 includes a heat flux detection unit 92CB and an acceptability determination unit 92CC. The heat flux detection unit 92CB detects the heat flux flowing from the component W1 through the solder S and then through the component W2 when the component W1 of the product U is heated at a temperature that does not melt the solder S, using the heat flux sensor 50. The acceptability determination unit 92CC determines whether the quality of the solder S is acceptable or unacceptable based on the heat flux detested by the heat flux detecting unit 92CB. The acceptability determination unit 92CC also executes processing according to the result of the determination. The result of the determination can be said to be an inspection result of the quality of the solder S.

According to the above configuration, the quality of the solder S is determined based on the heat flux detected by the heat flux sensor 50. Since the heat flux sensor 50 is generally capable of detecting heat flux with high sensitivity, with the above configuration, the heat flux sensor 50 can detect minute changes in the volume of voids, and the quality of the solder S can be determined with high accuracy. Therefore, the quality of the solder S can be inspected accurately. A similar technique would be to determine whether the solder S is acceptable or unacceptable based on the temperature detected by a thermocouple, etc., but the heat flux sensor 50 is more sensitive than a thermocouple, and therefore the quality of the solder S can be inspected accurately. Furthermore, since the heat flux sensor 50 has a high heat flux detection speed, the inspection time can be shortened.

In this embodiment, the heat flux detected by the heat flux detection unit 92CB is a plurality of heat fluxes (time-series data of heat flux in the above example; the same applies below.) detected at each of the plurality of timings, that is, the heat flux has a time variation. As described above, based on time variation, the acceptability determination unit 92CC may determine the quality of the solder S is unacceptable when the rate of increase of the heat flux is less than the threshold Th1, and determine the quality as acceptable when the rate increase is greater than or equal to the threshold Th1. This allows the quality of the solder S to be inspected accurately. The rate of increase of the heat flux used in the above determination is preferably the rate of increase during a period in which the heat flux increases rapidly. Example of such a period include (1) the period from start of heating of component W1 (product U) by heater tool 31 until the temperature of the heater tool 31 reaches the target heat generation temperature for the heating (period T1 to T4.5 in FIG. 8), or (2) the period from the start of heating until the heat flux reaches its peak value (period T1 to T4.5 in FIG. 9). The rate of increase may have a value of the rate of increase of the heat flux during a specified period, which may be all or part of these periods, divided by the specified period, or may have a differential value of the time variation in the heat flux at a certain timing within the periods. The same applies to the rate of increase below.

As described above, based on the time variation in the heat flux detected by the heat flux detection unit 92CB, the acceptability determination unit 92CC may determine that the quality is unacceptable when the rate of increase in the heat flux is less than the threshold Th1 and/or when the value of the heat flux at a predetermined timing is less than the threshold Th2, and determine the quality as acceptable in other cases. This allows the quality of the solder S to be inspected accurately.

As described above, based on the time variation in the heat flux detected by the heat flux detection unit 92CB, the acceptability determination unit 92CC may determine the quality to be unacceptable when the magnitude of the deviation between the pattern of this time variation (e.g., a graph of the time variation in the heat flux based on time-series data of the heat flux) and the pattern of the time variation in the heat flux when the quality of the solder S is acceptable, which has been prepared in advance, is equal to or greater than a predetermined reference amount, and determine the quality to be acceptable when the magnitude of the deviation is less than the predetermined reference amount. This allows the quality of the solder S to be inspected accurately.

In a modification example of this embodiment, the acceptability determination unit 92CC may function as an artificial intelligence model that receives the time variation in heat flux detected by the heat flux detection unit 92CB as input and outputs the acceptability of the quality as determination result. Such a model may be generated by machine learning using the acceptability of the quality obtained through experiments, etc. and the time variation of heat flux (time-series data) as learning data. The time variation here may be time-series data, graph data based on the time-series data (for example, image data of a graph), etc.

In addition, in the above embodiment, the joint inspection system 10 includes a heater tool 31 for heating the component W1 (product U) by contacting the component W1 and generating heat; a heat flux sensor 50 which contacts the component W2; and a controller 90 which serves as an inspection device. Such a joint inspection system 10 can operate as an inspection system for inspecting at least the quality of the solder S.

As described above, the controller 90 may further include a heating execution unit 92CA which controls the current that causes the heater tool 31 to generate heat, thereby causing the heater tool 31 to perform heating by the pulse heating method. Heating by pulse heating allows precise temperature control of the heater tool 31, and as a result, the quality of the solder S can be inspected accurately. The heating does not have to be pulse heating. Regardless of the type of heating, when there are many voids, the rate of increase and peak of the heat flux at the beginning of heating will be less than when there are few voids, as explained above, and therefore the quality can be determined using the same way of thinking as above.

As described above, the heater tool 31 is preferably configured to heat, from the side of the component W1, the workpiece W including the components W1, W2 and the solder S before soldering, and to perform soldering to join the components W1 and W2 with the solder S. This allows the heater tool 31 to be used in common for soldering and inspection, and also makes it easier to inspect the quality of the solder S of each of a plurality of products U obtained by a plurality of soldering operations. Furthermore, the joint inspection system 10 can be easily incorporated in-line into a given production line. Separate heater tools may be prepared for soldering and inspection. The stages 21 and 22 may be a common stage, but the stage is often preheated during soldering, and in this case, the preheated stage needs to be cooled more during inspection. For this reason, it is preferable to have separate stages for soldering and inspection, such as stages 21 and 22.

As described above, the controller 90 may be provided with a machine learning unit 92E for performing machine learning using, as learning data, the soldering condition used during performing soldering using the heater tool 31 and the acceptability determination results (inspection results) of the quality of the solder S soldered under this soldering condition. The machine learning unit 92E updates the soldering condition stored in the storage 91 through such machine learning. This makes the soldering condition suitable for soldering.

As described above, the joint inspection system 10 may include a stage 22 for supporting the heat flux sensor 50; a temperature sensor 62 for detecting the temperature of the stage 22; and a cooling device 61 for cooling the stage 22. The controller 90 may be provided with a cooling control unit 92D for controlling the cooling device 61 such that the stage 22 is maintained at a predetermined temperature at all times (in particular, before and during inspection) based on the temperature detected using the temperature sensor 62. This allows the temperature difference between the heater tool 31 and the stage 22 to be set to or close to the desired temperature difference during inspection of the solder S, thereby preventing the relationship between the volume of voids and the heat flux from becoming unstable each time the inspection is performed. This allows the quality of the solder S in the product U to be inspected accurately. It should be noted that the stage 22 only needs to be at a predetermined temperature at least before heating during the quality inspection, and therefore the cooling device 61 may be controlled at least before inspection.

As described above, the joint inspection system 10 may include a stage 22 for supporting the heat flux sensor 50; and a temperature sensor 62 for detecting the temperature of the stage 22. The controller 90 may further include a heating execution unit 92CA for controlling the current that causes the heater tool 31 to generate heat, thereby causing the heater tool 31 to heat the component W1 (product U). In a modification example, the heating execution unit 92CA may be configured to control the current such that the difference between the temperature of the heater tool 31 and the temperature detected using the temperature sensor 62 becomes a predetermined temperature difference, instead of or in addition to the above. For example, the heating execution unit 92CA feedback controls the current that causes the heater tool 31 to generate heat, with the target temperature being the sum of the temperature of the heater tool 31 detected by the temperature sensor 34 and the predetermined temperature difference. For example, the heating execution unit 92CA may prepare a plurality of inspection heating conditions which differ from each other depending on the temperature detected by the temperature sensor 62, and perform heating using the inspection heating condition depending on the temperature detected by the temperature sensor 62. With the above configuration, the temperature difference between the heater tool 31 and the stage 22 can be set to the desired temperature difference during inspection of the solder S, thereby preventing the relationship between the volume of voids and the heat flux from becoming unstable each time the inspection is performed. This allows the quality of the solder S in the product U to be inspected accurately. Furthermore, by combining such a configuration with the cooling of the stage 22, the temperature difference between the heater tool 31 and the stage 22 during the inspection of the solder S can be stably set to a desired temperature difference.

The above embodiment also concerns a method for inspecting the quality of the solder S in the product U. This inspection method includes a heating step of heating a component W1 of a product U at a temperature that does not melt solder S; a detection step of detecting, using a heat flux sensor 50, a heat flux flowing from the component W1 through solder S and then to component W2 as a result of the heating step' and a determination step of determining whether quality is acceptable or unacceptable based on the heat flux detected in the detection step. Each step can be performed by any entity (the same applies to the respective steps below). The quality of the solder S in the product U can be inspected accurately.

The heat flux detected in the detection step may have a time variation or a value at a certain timing. In the former case, the determination step can include a step of determining whether the quality is acceptable or unacceptable based on the time variation detected in the detection step. This allows the quality of the solder S in the product U to be inspected accurately.

In the heating step, the component W1 is heated by the heater tool 31 that has been used for soldering to join the components W1 and W2 with the solder S. This allows the heater tool to be shared between the soldering and inspection.

At least before the heating step, a cooling step may be performed for cooling the stage 22 supporting the heat flux sensor 50 to a predetermined temperature. With this configuration, the temperature difference between the heater tool 31 and the stage 22 can be set to or close to the desired temperature difference during inspection of the solder S, thereby preventing the relationship between the volume of voids and the heat flux from becoming unstable each time the inspection is performed.

The above embodiment also concerns a method for manufacturing a product U, that is, a soldered product. The manufacturing method includes a first step of producing a product U including components W1 and W2 with the solder S; and a second step of inspecting quality of the solder S of the produced product U (determining whether the quality is acceptable or unacceptable) according to the inspection method described above. This allow the product U to be obtained with a highly accurate quality inspection.

The manufacturing method further includes a third step of supplying to a subsequent process the product U when quality of the product U is determined to be acceptable by the inspection method in the second step, and not supplying the product U to the subsequent process when the quality of the product U is determined to be unacceptable by treating the product U as an unacceptable product. As a result, the product U that has been determined to be acceptable is supplied to the subsequent process with high accuracy.

### Modifications

Various modifications are possible to the above embodiment. For example, various configurations described in the above embodiments are optional and can be modified as appropriate. For example, the processor 92 may be constituted by one or more processing circuits such as one or more CPUs, one or more application specific integrated circuits (ASICs), and one or more field-programmable gate arrays (FPGAs), or a combination of a plurality of processing circuits. The processor 92 can also be called a processing unit. The program may be stored in a computer-readable non-transitory storage medium such as the non-volatile storage 91.

### Appendix

Configurations will be additionally described below as examples, each of which is based on at least part of the above-described embodiment(s) and modification(s). The configurations recited in Appendices can be combined with each other.

### Appendix 1

An inspection device configured to inspect quality of solder joining a first component and a second component, the inspection device comprising:

a detection unit configured to detect, by a heat flux sensor, a heat flux corresponding to a heat flow from the first component through the solder and then through the second component as a result of heating the first component at a temperature that does not melt the solder; and

an acceptability determination unit configured to determine whether the quality is acceptable or unacceptable based on the heat flux detected by the detection unit.

### Appendix 2

The inspection device according to Appendix 1, wherein the heat flux has a time variation; and the acceptability determination unit is configured to determine, based on the time variation, that the quality is unacceptable when a rate of increase of the heat flux is less than a first threshold and to determine, based on the time variation, that the quality is acceptable when the rate of increase is equal to or greater than the first threshold.

### Appendix 3

The inspection device according to Appendix1 or 2, wherein the heat flux has a value at a predetermined timing; and the acceptability determination unit is configured to determine that the quality is unacceptable when the value is less than a second threshold, and to determine that the quality is acceptable when the value is equal to or greater than the second threshold.

### Appendix 4

The inspection device according to Appendix 1, wherein the heat flux has a time variation; and the acceptability determination unit is configured to determine, based on the time variation, that the quality is unacceptable when at least one of the rate of increase of the heat flux being less than a third threshold and the value of the heat flux at a predetermined timing being less than a fourth threshold is satisfied, and to determine, based on the time variation, that the quality is acceptable in other cases.

### Appendix 5

The inspection device according to any one of Appendices 1 to 3, wherein the heat flux has a time variation; and the acceptability determination unit is configured to determine, based on the time variation, that the quality is unacceptable when a magnitude of deviation between a pattern of the time variation and a pattern of a previously prepared time variation in a heat flux when the quality is acceptable is equal to or greater than a predetermined reference amount, and to determine, based on the time variation, that the quality is acceptable when the magnitude of the deviation is less than the predetermined reference amount.

### Appendix 6

The inspection device according to any one of Appendices 1 to 5, wherein the heat flux has a time variation; and the acceptability determination unit is configured to function as an artificial intelligence model configured to receive the time variation as an input and to output whether the quality is acceptable or unacceptable as a determination result.

### Appendix 7

The inspection device according to any one of Appendices 1 to 6, comprising a processor configured to operate as the detection unit and the acceptability determination unit. The processor may also be configured to operate as at least one other unit of the inspection device (for example, each of the units described below).

### Appendix 8

An inspection system comprising: the inspection device according to any one of Appendices 1 to 7; a heater tool configured to heat the first component by generating heat while contacting the first component; and the heat flux sensor configured to contact the second component.

### Appendix 9

The inspection system according to Appendix 8, wherein the inspection device further includes a heating execution unit configured to control a current that generates heat in the heater tool to cause the heater tool to perform pulse heating as the heating.

### Appendix 10

The inspection system according to Appendix 8 or 9, wherein the heater tool heats a workpiece including the first component, the second component, and solder before soldering from a side of the first component, and performs soldering to join the first component and the second component with the solder.

### Appendix 11

The inspection system according to Appendix 10, wherein the inspection device further includes a machine learning unit configured to update a soldering condition by performing machine learning using as learning data the soldering condition when soldering is performed by the heater tool and a detemination result of acceptability of the quality of the solder under the soldering condition.

### Appendix 12

The inspection system according to any one of Appendices 8 to 11, further including: a stage configured to support the heat flux sensor; a temperature sensor configured to detect a temperature of the stage; and a cooling device configured to cool the stage, wherein the inspection device includes a cooling control unit configured to control the cooling device based on the temperature detected by the temperature sensor such that the stage reaches a predetermined temperature at least before the heating.

### Appendix 13

The inspection system according to any one of Appendices 8 to 12, further comprising: a stage configured to support the heat flux sensor; and a temperature sensor configured to detect a temperature of the stage, wherein the inspection device further includes a heating execution unit configured to control a current that causes the heater tool to generate heat, thereby causing the heater tool to perform the heating; and the heating execution unit is configured to control the current such that a difference between the temperature of the heater tool and the temperature detected by the temperature sensor becomes a predetermined temperature difference.

### Appendix 14

A non-transitory computer-readable storage medium storing a program that, when executed by a computer for inspecting quality of solder joining a first component and a second component, causes the computer to perform: a heating step of heating the first component at a temperature that does not melt the solder; a detection step of detecting, by a heat flux sensor, a heat flux corresponding to a heat flow from the first component through the solder and then through the second component as a result of the heating step; and a determination step of determining whether the quality is acceptable or unacceptable based on the heat flux detected by the detection step.

### Appendix 15

A method for inspecting quality of solder joining a first component and a second component, the method comprising the steps of a heating step of heating the first component at a temperature that does not melt the solder; a detection step of detecting, by a heat flux sensor, a heat flux corresponding to a heat flow from the first component through the solder and then through the second component as a result of the heating step; and a determination step of determining whether the quality is acceptable or unacceptable based on the heat flux detected by the detection step.

### Appendix 16

The inspection method according to Appendix 15, wherein the heat flux detected in the detection step has a time variation; and the determination step includes a step of determining whether the quality is acceptable or unacceptable based on the time variation.

### Appendix 17

The inspection method according to Appendix 15 or 16, wherein in the heating step, the first component is heated by a heater tool that has been used for soldering to join the first component and the second component with the solder.

### Appendix 18

The inspection method according to any one of Appendices 15 to 17, further comprising a cooling step of cooling a stage supporting the heat flux sensor to a predetermined temperature at least before the heating step.

### Appendix 19

a first step of producing a soldered product including the first component, the second component, and the solder by soldering the first component and the second component together with the solder; and a second step of inspecting quality of the solder in the produced soldered product by the inspection method according to any one of Appendices 15 to 18.

### Appendix 20

The method according to Appendix 19, further comprising a third step of supplying the soldered product to a subsequent process when the quality is determined to be acceptable by the inspection method in the second step, and not supplying the soldered product to the subsequent process when the quality is determined to be unacceptable by treating the soldered product as an unacceptable product.

### Scope of the present disclosure

The present disclosure has been described above according to the embodiments and modifications. However, the present disclosure is not limited to the above-described embodiments and modifications. For example, the present disclosure includes various modifications to the above- described embodiments and modifications that can be understood by those skilled in the art within the scope of the technical concept of the present disclosure. The configurations described in the above embodiments and modifications can be combined as appropriate as long as there is no inconsistency. Furthermore, components may be omitted in the configurations as appropriate.

### Incorporation by Reference

This application claims the benefit of Japanese Patent Application No. 2025-006077, filed on January 16, 2025, the entire disclosure of which is incorporated by reference herein.

### REFERENCE SYMBOLS LIST

10: joint inspection system; 21A: through-hole; 22: stage; 22A: recess; 23: transport device; 23A: holding member; 23B: driving device; 31: heater tool; 32: power supply circuit; 33: driving device; 34: temperature sensor; 40: ultrasonic application device; 41: oscillator; 42: horn; 43: driving device; 50: heat flux sensor; 61: cooling device; 62: temperature sensor; 70: input/output device; 90: controller; 91: storage; 92: processor; 92A: soldering execution unit; 92B: transport execution unit; 92C: quality inspection execution unit; 92CA: heating execution unit; 92CB: heat flux detection unit; 92CC: acceptability determination unit; 92D: cooling control unit; 92E: machine learning unit; 93: main memory; HF: heat flow; S: solder; U: product; W: workpiece; W1: component; W2: component

1. An inspection device configured to inspect quality of solder j oining a first component and a second component, the inspection device comprising:
a detection unit configured to detect, by a heat flux sensor, a heat flux corresponding to a heat flow from the first component through the solder and then through the second component as a result of heating the first component at a temperature that does not melt the solder; and
an acceptability determination unit configured to determine whether the quality is acceptable or unacceptable based on the heat flux detected by the detection unit.

2. The inspection device according to claim 1, wherein
the heat flux has a time variation; and
the acceptability determination unit is configured to determine, based on the time variation, that the quality is unacceptable when a rate of increase of the heat flux is less than a first threshold and to determine, based on the time variation, that the quality is acceptable when the rate of increase is equal to or greater than the first threshold.

3. The inspection device according to claim 1, wherein
the heat flux has a value at a predetermined timing; and
the acceptability determination unit is configured to determine that the quality is unacceptable when the value is less than a second threshold, and to determine that the quality is acceptable when the value is equal to or greater than the second threshold.

4. The inspection device according to claim 1, wherein
the heat flux has a time variation; and
the acceptability determination unit is configured to determine, based on the time variation, that the quality is unacceptable when at least one of the rate of increase of the heat flux being less than a third threshold and the value of the heat flux at a predetermined timing being less than a fourth threshold is satisfied, and to determine, based on the time variation, that the quality is acceptable in other cases.

5. The inspection device according to claim 1, wherein
the heat flux has a time variation; and
the acceptability determination unit is configured to determine, based on the time variation, that the quality is unacceptable when a magnitude of deviation between a pattern of the time variation and a pattern of a previously prepared time variation in a heat flux when the quality is acceptable is equal to or greater than a predetermined reference amount, and to determine, based on the time variation, that the quality is acceptable when the magnitude of the deviation is less than the predetermined reference amount.

6. The inspection device according to claim 1, wherein
the heat flux has a time variation; and
the acceptability determination unit is configured to function as an artificial intelligence model configured to receive the time variation as an input and to output whether the quality is acceptable or unacceptable as a determination result.

7. The inspection device according to claim 1, comprising
a processor configured to operate as the detection unit and the acceptability determination unit.

8. An inspection system comprising:
the inspection device according to claim 1;
a heater tool configured to heat the first component by generating heat while contacting the first component; and
the heat flux sensor configured to contact the second component.

9. The inspection system according to claim 8, wherein
the inspection device further includes a heating execution unit configured to control a current that generates heat in the heater tool to cause the heater tool to perform pulse heating as the heating.

10. The inspection system according to claim 8, wherein
the heater tool heats a workpiece including the first component, the second component, and solder before soldering from a side of the first component, and performs soldering to join the first component and the second component with the solder.

11. The inspection system according to claim 10, wherein
the inspection device further includes a machine learning unit configured to update a soldering condition by performing machine learning using as learning data the soldering condition when soldering is performed by the heater tool and a determination result of acceptability of the quality of the solder under the soldering condition.

12. The inspection system according to claim 8, further including:
a stage configured to support the heat flux sensor;
a temperature sensor configured to detect a temperature of the stage; and
a cooling device configured to cool the stage, wherein the inspection device includes a cooling control unit configured to control the cooling device based on the temperature detected by the temperature sensor such that the stage reaches a predetermined temperature at least before the heating.

13. The inspection system according to claim 8, further comprising:
a stage configured to support the heat flux sensor; and
a temperature sensor configured to detect a temperature of the stage, wherein the inspection device further includes a heating execution unit configured to control a current that causes the heater tool to generate heat, thereby causing the heater tool to perform the heating; and
the heating execution unit is configured to control the current such that a difference between the temperature of the heater tool and the temperature detected by the temperature sensor becomes a predetermined temperature difference.

14. A non-transitory computer-readable storage medium storing a program that, when executed by a computer for inspecting quality of solder joining a first component and a second component, causes the computer to perform:
heating the first component at a temperature that does not melt the solder;
detecting, by a heat flux sensor, a heat flux corresponding to a heat flow from the first component through the solder and then through the second component as a result of the heating; and
determining whether the quality is acceptable or unacceptable based on the detected heat flux.

15. A method for inspecting quality of solder joining a first component and a second component, the method comprising:
heating the first component at a temperature that does not melt the solder;
detecting, by a heat flux sensor, a heat flux corresponding to a heat flow from the first component through the solder and then through the second component as a result of the heating; and
determining whether the quality is acceptable or unacceptable based on the detected heat flux.

16. A manufacturing method comprising:
producing a soldered product including the first component, the second component, and the solder by soldering the first component and the second component together with the solder; and
inspecting quality of the solder in the produced soldered product by the inspection method according to claim 15.
